(19) United States
(12) Patent Application Publication
(10) Pub. No.: US 2019/0022055 A1
(43) Pub. Date: Jan. 24, 2019

(54) FOOD BASED DELIVERY OF CANNABINOIDS

(71) Applicants: Corey A. SIEGEL, Newton, MA (US); Joshua KORZENIK, Newton, MA (US); Michael ALLIO, Newton, MA (US); ColonaryConcepts LLC, Palm Beach Gardens, FL (US)

(72) Inventors: Corey A. SIEGEL, Palm Beach Gardens, FL (US); Joshua KORZENIK, Palm Beach Gardens, FL (US); Michael K. ALLIO, Palm Beach Gardens, FL (US); Herbert B. STERN, Palm Beach Gardens, FL (US)

(21) Appl. No.: 16/067,765

(22) PCT Filed: Jan. 6, 2017
(86) PCT No.: PCT/US17/12622

§ 371 Date: Jul. 2, 2018

Related U.S. Application Data
(60) Provisional application No. 62/276,687, filed on Jan. 8, 2016.
(60) Provisional application No. 62/276,685, filed on Jan. 8, 2016.

(51) Int. Cl. A61K 31/353; A23L 33/00

(52) U.S. Cl. CPC A61K 31/353; A23L 33/105; A23L 33/40

(57) ABSTRACT

Described herein are compositions comprising one or more United States Pharmacopeial Convention (USP) grade food ingredients mixed together with a cannabinoid. In some embodiments of the compositions and methods described herein, the compositions comprises a mixture which comprises a food item or a meal containing cannabinoid.

### CROSS-REFERENCE

This application claims the benefit of U.S. Provisional Application No. 62/276,687, filed Jan. 8, 2016 and U.S. Provisional Application No. 62/276,685, filed Jan. 8, 2016, which applications are both incorporated herein by reference.

### BACKGROUND

Cannabinoids comprise chemical compositions that act on cannabinoid receptors. Cannabinoids act on cannabinoid receptors so that these receptors affect neurotransmitter release.

Cannabinoids include the endocannabinoids, which is produced naturally in the body by humans and animals, the phytocannabinoids, which is found in the cannabis plant and some other plants, and synthetic cannabinoids, which are manufactured artificially. The cannabis plant includes the tetrahydrocannabinol (THC), which is a psychoactive composition. Marijuana typically refers to the dried flowers, leaves, and stems of the cannabis plant. Marijuana is used both recreationally and medicinally.

### SUMMARY

Described herein are compositions and methods that comprise formulations including one or more United States Pharmacopeial Convention (USP) or USP-NF grade food ingredients that provide a delivery vehicle for an oral active agent such as a cannabinoid.

A USP-NF grade ingredient or chemical comprises an ingredient or chemical that meets the standards as listed in monographs found in the United States Pharmacopeia and the National Formulary (USP-NF). A monograph includes the name of the ingredient or preparation, the definition, packaging, storage, and labeling requirements, and the specification. Generally, ingredients that are of USP-NF grade are at a relatively high level of purity as compared to non-USP-NF grade ingredients.

The formulations that are utilized in the compositions and methods described herein provide numerous beneficial properties. For example, USP-NF grade food ingredients contain less contaminant than typical food ingredients and as such a formulation of cannabinoids with USP-NF grade food ingredients produces less cannabinoid degradation products than were the cannabinoids mixed with typical non-USP-NF food ingredients. Thus, for example, the cannabinoids maintain a higher potency over time in the formulations described herein as compared to a mixture of cannabinoid with non-USP-NF food ingredients. For example, USP-NF grade food ingredients contain less contaminant than typical food ingredients and as such the formulations described herein will have longer a longer as compared to a mixture of cannabinoid with non-USP-NF food ingredients.

In addition, because they contain relatively fewer contaminants, USP-NF grade food ingredients have properties that are much more consistent and predictable than non-USP-NF grade food ingredients. The increased consistency and predictability of USP-NF grade food ingredients provides the ability to tailor the delivery of the cannabinoid within the formulation. For example, in some embodiments of the composition, the composition is configured to deliver (i.e. release) the cannabinoid at a defined location within the GI tract. For example, in some embodiments of the composition, the composition is configured to deliver (i.e. release) the cannabinoid over a pre-defined rate of release and/or over a pre-defined period of time.

Certain embodiments of the formulations that are utilized in the compositions and methods described herein are configured so that their components work synergistically to enhance the benefits offered by the different components of the formulation. For example, certain cannabinoid types are associated with antimicrobial properties. The antimicrobial properties of the cannabinoid are enhanced by the relatively pure USP-NF grade food ingredients that the cannabinoid is mixed with to provide an enhanced antimicrobial activity that, for example, prolongs shelf life. For example, certain cannabinoid types are associated with immune enhancement and when mixed with an immune enhancing food ingredient such as, for example, ginger, the overall immune enhancement of the formulation is increased.

In addition, typically, the traditional edible cannabinoid product does not provide a convenient form for delivering a sufficient dose of cannabinoid to an individual. In some embodiments of the compositions and methods described herein, a cannabinoid is provided in a food bar comprising USP N-F grade food ingredients, which provides a convenient portable vehicle for delivering an accurate and sufficient amount of cannabinoid to an individual who ingests said food bar. Certain cannabinoids are associated with cognitive impairment. Because a sufficient dose is provided within the food bars described herein, an individual will, for example, be prevented from taking an improper amount due to cognitive impairment (i.e. because the entire sufficient dose is pre-measured and contained within the food bar).

Described herein is a composition, comprising one or more United States Pharmacopeial Convention (USP) grade food ingredients and a cannabinoid; wherein the composition comprises a mixture of the one or more USP grade food ingredients and the cannabinoid. In some embodiments of the compositions and methods described herein, the mixture comprises a food bar. In some embodiments of the compositions and methods described herein, the mixture comprises a meal. In some embodiments of the compositions and methods described herein, the mixture is a homogenous mixture. In some embodiments of the compositions and methods described herein, the mixture is heated. In some embodiments of the compositions and methods described herein, the mixture is heated by mixing it with a heated binder. In some embodiments of the compositions and methods described herein, the binder comprises cocoa butter. In some embodiments of the compositions and methods described herein, the binder comprises coconut oil. In some embodiments of the compositions and methods described herein, the heated binder is heated to a temperature above 90 degrees Fahrenheit and the one or more therapeutic agents is mixed with the heated binder when it cools to a temperature below 80 degrees Fahrenheit. In some embodiments of the compositions and methods described herein, the cannabinoid comprises marijuana. In some embodiments of the compositions, the composition includes a pre-measured amount of cannabinoid.

Described herein is a method comprising providing to an individual a composition comprising a mixture of a cannabinoid and one or more USP grade food ingredients. In some embodiments of the method, the mixture comprises a food bar. In some embodiments of the method, the mixture comprises a meal. In some embodiments of the method, the mixture is a homogenous mixture. In some embodiments of the method, the mixture is heated. In some embodiments of the method, the mixture is heated by mixing it with a heated binder. In some embodiments of the method, the binder comprises cocoa butter. In some embodiments of the method, the binder comprises coconut oil. In some embodiments of the method, the heated binder is heated to a temperature above 90 degrees Fahrenheit and the one or more therapeutic agents is mixed with the heated binder when it cools to a temperature below 80 degrees Fahrenheit. In some embodiments of the method, the mixture comprises a pre-measured dose of cannabinoid.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

Described herein are compositions and methods that comprise formulations that provide modes of delivery of a cannabinoid to a subject. In some embodiments of the compositions or methods described herein, a composition comprises a cannabinoid mixed together with one or more United States Pharmacopeial Convention (USP) grade food ingredients to form an edible food or drink. In some embodiments of the compositions and methods described herein, the cannabinoid and the food ingredient form an essentially homogeneous mixture. In some embodiments of the compositions and methods described herein, the cannabinoid comprises a USP grade cannabinoid.

In some embodiments of the compositions and methods described herein, a composition as described herein comprises a mixture of a cannabinoid or a cannabinoid containing composition and one or more USP grade food ingredients. Non-limiting examples of suitable cannabinoids include tetrahydrocannabinol (THC), cannabigerol, cannabichromene, cannabicyclol, cannabivarin, tetrahydrocannabivarin, cannabidivarin, cannabichromevarin, cannabigerovarin, and cannabigerol monomethyl ether. Cannabinoids are found in a number of compositions. Non-limiting examples of cannabinoid containing compositions suitable for use with the compositions described herein include cannabis as well as synthetic cannabinoid containing compositions. Cannabis further comprises a number of forms. Non-limiting examples of cannabis forms include marijuana, hashish (a cannabis derived resign), and hashish oil (a cannabis derived resign derived from solvent extraction).

A USP-NF grade ingredient or chemical comprises an ingredient or chemical that meets the standards as listed in monographs found in the United States Pharmacopeia and the National Formulary (USP-NF). A monograph includes the name of the ingredient or preparation, the definition, packaging, storage, and labeling requirements, and the specification. The specification consists of a series of tests, procedures for the tests, and acceptance criteria. Any and all USP-NF grade listed foods are suitable for use with the compositions and methods described herein.

In some embodiments of the compositions and methods described herein, a cannabinoid is mixed together with one or more USP-NF grade food ingredients to form a food bar comprising one or more of any USP-NF grade food ingredients typically found in a food bar such as, for example, USP-NF grade chocolate and/or USP-NF grade nuts, and/or USP-NF grade fruit, and/or USP-NF grade sweetener thus forming a delivery modality comprising a bar.

In some embodiments of the compositions described herein, a USP-NF grade food ingredient comprises a sweetener. In some embodiments of the compositions and methods described herein, a USP-NF grade sweetener comprises granulated sucrose. In some embodiments of the compositions and methods described herein a USP-NF grade sweetener comprises a syrup. In some embodiments of the compositions and methods described herein, a USP-NF grade sweetener comprises invert syrup. In some embodiments of the compositions and methods described herein, a USP-NF grade sweetener comprises erythritol. In some embodiments of the compositions and methods described herein, a USP-NF grade sweetener comprises maltodextrin. In some embodiments of the compositions and methods described herein, a USP-NF grade sweetener comprises dextrin.

In some embodiments of the compositions and methods described herein, a USP-NF grade ingredient comprises a texturizer. In some embodiments of the compositions and methods described herein, a USP food ingredient comprises cocoa butter. In some embodiments of the compositions and methods described herein, a USP-NF grade ingredient comprises malic acid. In some embodiments of the compositions and methods described herein, a USP-NF grade ingredient comprises citric acid. In some embodiments of the compositions and methods described herein, a USP-NF grade ingredient comprises lemon oil. In some embodiments of the compositions and methods described herein, a USP-NF grade ingredient comprises vanilla flavor.

In some embodiments of the compositions and methods described herein, a composition as described herein may comprise a solid food containing a cannabinoid. In some embodiments of the compositions and methods described herein, a composition as described herein may comprise a liquid containing a cannabinoid.

Combining together or mixing of one or more cannabinoid and one or more USP-NF food ingredients is used to generate essentially any conceivable food, drink, candy, or gum. For example, compositions containing cannabinoids comprise meals such as, for example, soup, lasagna or chicken with rice. For example, compositions containing cannabinoids comprise shakes and flavored drinks. For example, compositions containing cannabinoids comprise cookies, brownies, and cakes.

In some embodiments of the compositions and methods described herein, a composition as described herein may comprise a bar containing a cannabinoid. In some embodiments of the compositions and methods described herein, a composition as described herein may comprise a shake containing a cannabinoid. In some embodiments of the compositions and methods described herein, a composition as described herein may comprise a smoothie containing a cannabinoid.

Described herein is a method for manufacturing a composition. In some embodiments of the compositions and methods described herein, the method comprises heating a binder to a temperature at which the fat becomes at least partially liquefied then mixing the at least partially liquefied fat with a cannabinoid.

In some embodiments of the compositions and methods described herein, the temperature to which the binder is heated comprises about 95 degrees Fahrenheit. In some embodiments of the compositions and methods described herein, the temperature to which the binder is heated comprises about 90 degrees Fahrenheit. In some embodiments of the compositions and methods described herein, the temperature to which the binder is heated comprises about 85 degrees Fahrenheit. In some embodiments of the compositions and methods described herein, the temperature to which the binder is heated comprises about 80 degrees Fahrenheit. In some embodiments of the compositions and methods described herein, the temperature to which the binder is heated comprises about 75 degrees Fahrenheit.

In some embodiments of the compositions and methods described herein, the heated binder cools to a temperature of about 80 degrees before a cannabinoid is mixed with it. In some embodiments of the compositions and methods described herein, the heated binder cools to a temperature of about 75 degrees before a cannabinoid is mixed with it. In some embodiments of the compositions and methods described herein, the heated binder cools to a temperature of about 70 degrees before a cannabinoid is mixed with it. In some embodiments of the compositions and methods described herein, the heated binder cools to a temperature of about 65 degrees before a cannabinoid is mixed with it. In some embodiments of the compositions and methods described herein, the heated binder cools to a temperature of about 60 degrees before a cannabinoid is mixed with it.

In some embodiments of the compositions and methods described herein, a composition comprises an essentially homogeneous mixture of a cannabinoid and a fat, wherein the fat is an essentially solid form at room temperature and an essentially liquid form at body temperature. In some embodiments of the compositions and methods described herein, the fat coats the composition by becoming an essentially liquid form when in mouth and pharynx of the subject which are at body temperature.

In some embodiments of the compositions and methods described herein, the compositions described herein comprise microencapsulated cannabinoids. Microencapsulation comprises coating small particles of a gas, a liquid, a solid, or a combination thereof with a coating. The coating shields the gas, liquid, solid, or combination thereof so that, for example, when a microencapsulated gas, liquid, solid, or combination thereof is ingested by an individual, the coating prevents the microencapsulated gas, liquid, solid, or combination from contacting the taste buds of the individual. Thus, the individual does not taste or experience the texture of the microencapsulated gas, liquid, solid, or combination thereof when ingested.

Non-limiting examples of coatings suitable for use with the compositions and methods described herein include ethyl cellulose, polyvinyl alcohol, gelatin, sodium alginate, and chitin.

In some embodiments of a method as described herein, the method comprises providing any of the compositions as described herein to an individual. In some embodiments of a method as described herein, the method comprises manufacturing a composition as described herein, wherein a heated binder is heated to a temperature above 90 degrees Fahrenheit, and the one or more therapeutic agents is mixed with the heated binder when the heated binder cools to a temperature below 80 degrees Fahrenheit.

In some embodiments of the compositions and methods described herein, a composition is configured to deliver a cannabinoid incorporated therein to a specific location within the gastrointestinal tract. The delivery of the cannabinoid within the gastrointestinal tract is related to the rate of digestion of the composition in which the cannabinoid is incorporated. For example, a composition comprising a thin edible layer of USP N-F grade food ingredients takes relatively less time to digest and is digested in the proximal part of the intestinal tract releasing the capsule in the proximal part of the intestinal tract. For example, a composition comprising a thick edible layer of a USP N-F takes relatively more time to digest and is digested in the distal portion of the digestive tract. Thus, by, for example, controlling the thickness of the composition, a point of release of the capsule within the gastrointestinal tract is controlled. Similarly, in some embodiments, the composition comprises of ingredients that are digested in the proximal portion of the gastrointestinal tract, and in some embodiments, the composition comprises ingredients that are digested in the distal portion of the gastrointestinal tract. Alternatively or additionally, a USP N-F grade food ingredient coating a cannabinoid may be configured to dissolve at a specific pH thus determining the location within the gastrointestinal tract that a cannabinoid is released. Similarly, formulations of the compositions described herein are used to provide accurate time-release of a cannabinoid through configuring the USP N-F grade food ingredients to so that they are digested at a certain rate or dissolve at a certain rate. Because of their relative purity, the digestion rates of USP N-F grade foods are accurately derivable and as such provide for accurate release at a particular location and/or at a particular time.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A composition, comprising
one or more United States Pharmacopeial Convention and the National Formulary (USP-NF) grade food ingredients; and
a cannabinoid;
wherein the composition comprises a mixture of the one or more USP-NF grade food ingredients and the cannabinoid.

2. The composition of claim 1, wherein the mixture comprises a food bar.

3. The composition of claim 1, wherein the mixture comprises a shake.

4. The composition of claim 1, wherein the mixture comprises a meal.

5. The composition of claim 1, wherein the mixture is a homogenous mixture.

6. The composition of claim 1, wherein the mixture is heated.

7. The composition of claim 1, wherein the mixture is heated by mixing it with a heated binder.

8. The composition of claim 7, wherein the binder comprises cocoa butter.

9. The composition of claim 7, wherein the heated binder is heated to a temperature above 90 degrees Fahrenheit and the one or more therapeutic agents is mixed with the heated binder when it cools to a temperature below 80 degrees Fahrenheit.

10. The composition of claim 1, wherein the composition comprises a pre-measured dose of cannabinoid.

11. The composition of claim 1, wherein the cannabinoid comprises marijuana.

12. A method, comprising
providing to an individual a composition comprising a mixture of a cannabinoid and one or more USP grade food ingredients; and
wherein the heated binder is heated to a temperature above 90 degrees Fahrenheit and the one or more therapeutic agents is mixed with the heated binder when the heated binder cools to a temperature below 80 degrees Fahrenheit.

13. The method of claim 12, wherein the mixture comprises a food bar.

14. The method of claim 12, wherein the mixture comprises shake.

15. The method of claim 12, wherein the mixture comprises a meal.

16. The method of claim 12, wherein the mixture is a homogenous mixture.

17. The method of claim 12, wherein the mixture is heated.

18. The method of claim 12, wherein the mixture is heated by mixing it with a heated binder.

19. The method of claim 18, wherein the binder comprises cocoa butter.

20. The method of claim 12, wherein the mixture comprises a pre-measured dose of cannabinoid.
